(19) United States
(12) Patent Application Publication
(10) Pub. No.: US 2013/0098364 A1
(43) Pub. Date: Apr. 25, 2013

(54) PILLOW FOR USE WITH ASSISTED BREATHING MASKS

(71) Applicants: Edmund Scott Davis, Oakland Park, FL (US); Keith Richard Coponi, Oakland Park, FL (US)

(72) Inventors: Edmund Scott Davis, Oakland Park, FL (US); Keith Richard Coponi, Oakland Park, FL (US)

(73) Assignee: Banyan Licensing, L.L.C., Oakland Park, FL (US)

(21) Appl. No.: 13/269,557

(22) Filed: Oct. 7, 2011

Related U.S. Application Data
(60) Provisional application No. 61/390,924, filed on Oct. 7, 2010.

(51) Int. Cl. A61M 16/06; A47G 9/10

(52) U.S. Cl. CPC A61M 16/06; A47G 9/10
USPC 128/206.21; 5/636

(57) ABSTRACT

A pillow is provided for use with a respiratory mask, comprising first and second members, wherein the first member is made from a first material and the second member is made from a second material and wherein the first material is different from the second material. The first member or second member may define a plurality of apertures extending at least partially therethrough.

### FIELD OF THE INVENTION

The present invention relates to a pillow for use with assisted breathing masks to facilitate resting and sleep.

### BACKGROUND OF THE INVENTION

Users of respiratory masks often have difficulty resting or sleeping because pressure on the mask exerted by a pillow while side sleeping can impede the performance and comfort of the mask, cause the mask seal to separate from the face and result in air leakage (which can cause irritation from the eyes drying out), and/or cause the mask to press uncomfortably against the user's face. Users of respiratory masks often try to compensate for leaks and positional problems by tightening the mask against the face more than otherwise required, causing further discomfort. The ultimate effect of these problems is sleep interruption or non-compliance by the user (i.e., the user stops using the mask).

### SUMMARY OF THE INVENTION

The present invention relates to a pillow for use with assisted breathing masks to facilitate resting and sleep.

In one embodiment, the present invention comprises a pillow for use with a respiratory mask, comprising first and second members, wherein the first member is made from a first material and the second member is made from a second material and wherein the first material is different from the second material. In one embodiment, the first material and second material are a urethane foam and wherein the first material has a lower density than the second material. In another embodiment, the first material and second material are a urethane foam and wherein the first material has a greater indentation load deflection than the second material. In another embodiment, the first member is formed of high resilient urethane foam. In another embodiment, the second member is formed of a memory urethane foam. In another embodiment, the first member defines a plurality of apertures at least partially therethrough. In another embodiment, the second member defines a plurality of apertures at least partially therethrough. In yet another embodiment, the first member and second member have substantially the same configuration.

In one embodiment, the pillow comprises an intermediate member positioned between the first member and the second member. In another embodiment, the intermediate member is formed of a third material that is the same as one of the first material or the second material. In another embodiment, the intermediate member is formed of a third material that is different from the first material and the second material. In another embodiment, the intermediate member is formed of a urethane foam. In another embodiment, the intermediate member defines a plurality of apertures at least partially therethrough. In another embodiment, the intermediate member and at least one of the first member and second member have substantially the same configuration.

In one embodiment, the pillow comprises a cover that at least partially covers the first and second members. In another embodiment, at least a portion of the cover is formed of a spaced fabric. In another embodiment, the cover comprises a fiberfilled surface on at least one side. In another embodiment, the fiberfilled surface is positioned adjacent the first member. In still another embodiment, the cover comprises a hose tether extending therefrom.

In one embodiment, the present invention includes a method of using a respiratory mask comprising providing a pillow having first and second members, wherein the first member is made of a first material and the second member is made from a second material and wherein the first material is different from the second material; positioning a respiratory mask on the head of a user; and supporting the user's head on the pillow.

Thus, there is provided a pillow for use with a respiratory mask that provides a sleep surface choice to match a user's preference, heat reduction for cool sleeping, reduced perspiration for dry comfort and orthopedic design for proper head, neck and spine alignment.

### BRIEF DESCRIPTION OF THE DRAWING

Reference will now be made to the accompanying drawings, which are not necessarily drawn to scale, and wherein:

FIG. 1 is a perspective view illustrating a pillow, according to one embodiment of the invention.

FIG. 2A is a partial-cut-away view illustrating the pillow of FIG. 1;

FIG. 2B is a partial-sectional view illustrating a spacer fabric, also referred to as a three dimensional (3-D) mesh, used to construct the cover, according to one embodiment of the present invention;

FIG. 3A is a partial-cut-away view illustrating the pillow of FIG. 1;

FIG. 3B is a partial-sectional view illustrating a spacer fabric used to construct the cover, according to one embodiment of the present invention;

FIG. 3 is a partial-exploded view illustrating the pillow of FIG. 1 without a cover;

FIG. 4 is a side-elevation view illustrating the pillow of FIG. 3;

FIG. 5 is a plan view illustrating the pillow of FIG. 4;

FIG. 6 are plan views illustrating users with conventional pillows labeled “before” views and black and white plan views illustrating users with the pillow of FIG. 1; and

FIG. 7 is a perspective view of a spacer fabric, also referred to as a three dimensional (3-D) mesh, used to construct the cover, according to one embodiment of the present invention.

### DETAILED DESCRIPTION OF THE INVENTION

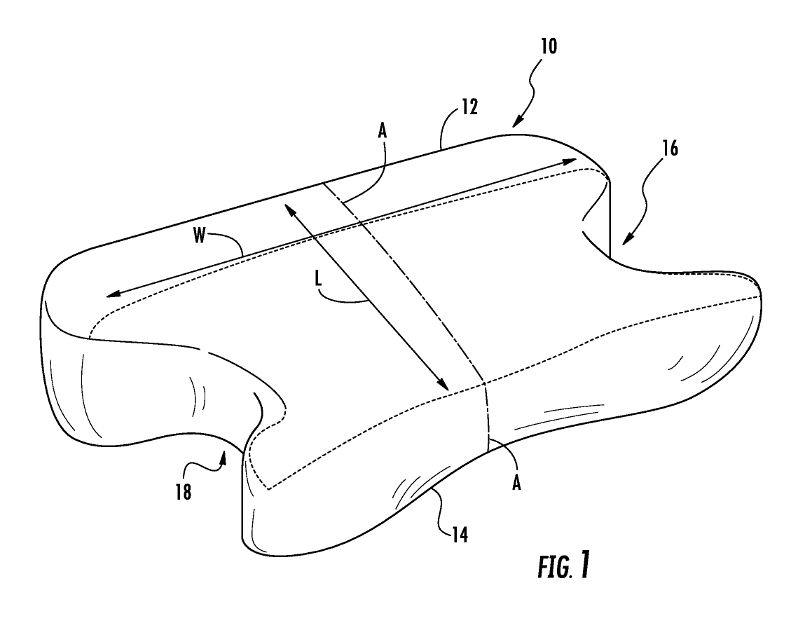

Referring to FIG. 1, there is illustrated a pillow 10 according to one embodiment of the present invention, for use with assisted breathing or respiratory masks, such as continuous positive airway pressure (“CPAP”) masks, auto adjusting positive airway pressure (“APAP”) masks, dual positive airway pressure (“BiPAP”) masks, and demand positive airway pressure (“DPAP”) masks, and other mask types for oxygen, etc., to facilitate resting and sleep

The configuration of the pillow 10 may vary. In the embodiment illustrated in FIG. 1, the pillow 10 is generally rectangular in shape and includes a rear side 12, a front side 14, a first lateral side 16, and a second lateral side 18. In one embodiment, the pillow 10 has an axis of symmetry “A” extending between the rear side 12 and front side 14. The pillow 10 has a width “W” extending between the first lateral side 16 and the second lateral side 18 and a length “L” extending between the rear side 12 and the front side 14. The invention is not limited to any particular shape for the pillow 10.

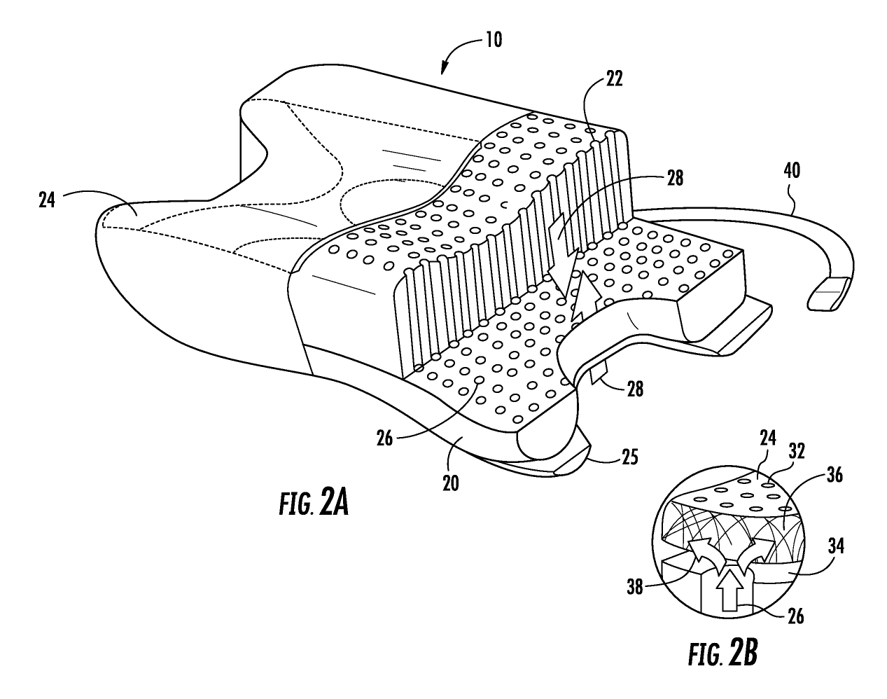

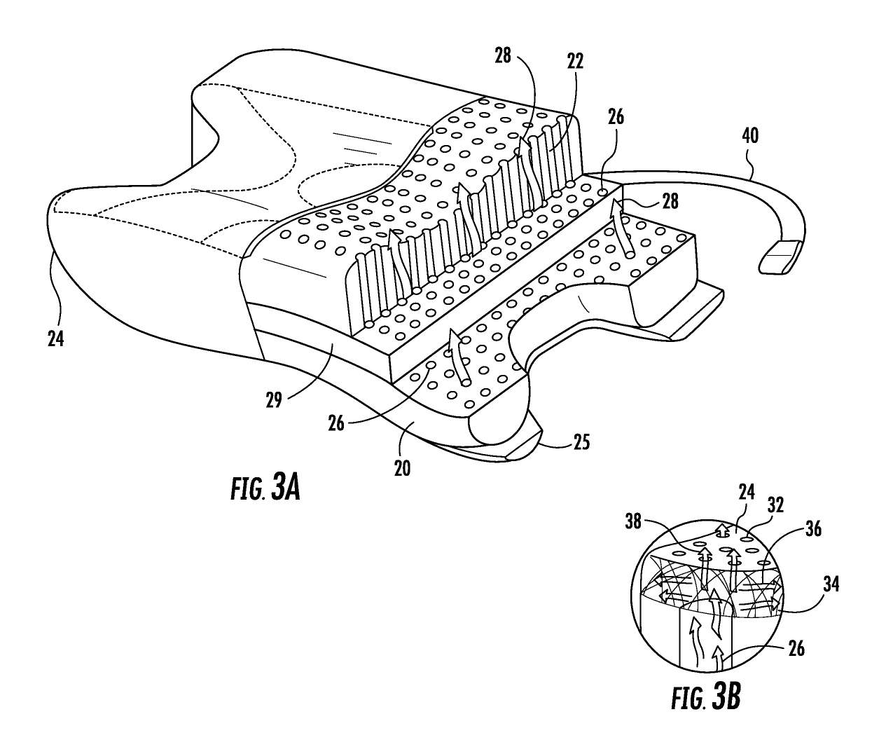

Referring to FIG. 2A, in one embodiment, the pillow 10 has a first member 20, a second member 22, and a cover 24 that at least partially covers the first member and second member. The first member 20 of the pillow 10 may be constructed of a variety of materials, including a polymer foam, gel, fiber fill inside a casing, and fluid inside a bladder. In one embodiment, the first member 20 is constructed of a high resilient (“HR”) urethane foam. According to one embodiment of the present invention, the first member 20 is constructed of a urethane foam has a density of 10 to 32. The density is expressed as a two digit number and there is actually a decimal between the two numbers which makes the density rating of the #10 foam a 1.0 foam and the density rating of a #32 foam a 3.2 foam. In the production of foam, there are two basic ingredients which are the urethane chemical that is the foam, and air. The more chemical that exists in the foam material, the higher its density rating will be. In the above range, the 1.0 rating means that there is 1.0 pounds of urethane chemical in each cubic foot of foam material and the 3.2 rating means that there is 3.2 pounds of urethane chemical in each cubic foot of foam material. A cubic foot is equal to an area that is 12″×12″×12″. According to one embodiment of the present invention, the urethane foam has an indentation load deflection (“ILD”) rating of 11 to 30. The ILD is a measure of how much pressure it takes to compress the foam twenty-five percent (25%) of its thickness. For instance, if a pillow is 4″ thick and has an ILD rating of 33, it would take 33 pounds of pressure to condense the foam to a thickness of 3″. In another embodiment, the urethane foam has an ILD of 12 to 26. In another embodiment, the urethane foam has an ILD of 14 to 24. In another embodiment, the urethane foam has an ILD of 16 or 17 to 20.

The second member 22 of the pillow 10 may be constructed of a variety of materials, including a polymer foam, gel, fiber fill inside a casing, and fluid inside a bladder. In one embodiment, the second member 22 of the pillow 10 is constructed of a urethane foam. According to one embodiment of the present invention, the urethane foam has a density of 18 to 60. As noted above, the density is expressed as a two digit number and there is actually a decimal between the two numbers which makes the density rating of the #18 foam a 1.8 foam and the density rating of a #60 foam a 6.0 foam. In the above range, the 1.8 rating means that there is 1.8 pounds of urethane chemical in each cubic foot of foam material and the 6.0 rating means that there is 6.0 pounds of urethane chemical in each cubic foot of foam material. According to one embodiment of the present invention, the urethane foam has an indentation load deflection (“ILD”) rating of 6 to 21. In another embodiment, the urethane foam has an ILD of 7 or 8 to 17. In another embodiment, the urethane foam has an ILD of 12 to 17. In another embodiment, the urethane foam has an ILD of 12 to 15. In one embodiment the urethane foam used to construct the second member 22 is a memory urethane foam.

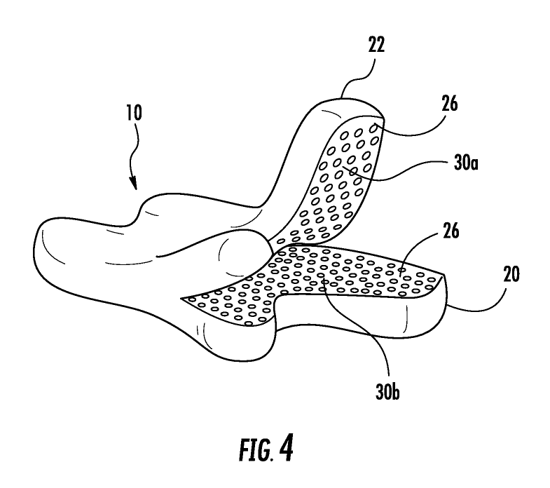

In another embodiment, illustrated in FIG. 3A, the pillow 10 includes an intermediate member 29 between the first member 20 and the second member 22. The intermediate member 29 is primarily for modifying the thickness of the pillow 10, but can also be used to increase or decrease the firmness of the pillow 10. The intermediate member 29 of the pillow 10 may be constructed of a variety of materials, including a polymer foam, gel, fiber fill inside a casing, and fluid inside a bladder. In one embodiment, the intermediate member 29 has the same density and ILD as the HR foam of the first member 20. In another embodiment, the intermediate member 29 has the same density and ILD as the memory foam of the second member 30. In another embodiment, the intermediate member can be formed of a urethane foam that is different than the urethane foam used to form the first member 20 and the second member 22. In one embodiment, the urethane foam used to form the intermediate member 29 has a density of between 10 to 60 and an ILD of 6 to 40.

As illustrated in FIG. 2A, in one embodiment, the first member 20 and/or the second member 22 of the pillow 10 are perforated such that the sides define a plurality of apertures 26 therethrough or at least partially therethrough. The apertures 26 enable air to flow into and, where the apertures extend through the first member 20 and/or second member 22 of the pillow 10, through the first member 20 and/or second member 22 of the pillow 10 to thereby ventilate the pillow with cool air. As illustrated in FIG. 2A, the apertures 26 extend through the first member 20 and the second member 22 of the pillow 10 such that air flow therethrough as illustrated by the arrows 28. As illustrated in FIG. 3A, in embodiments of the pillow 10 containing the intermediate member 29, the intermediate member 29 may also be perforated (although in some embodiments the intermediate member may not be perforated) so as to define a plurality of apertures 26 therethrough, or at least partially therethrough, to enable air to flow into and, where the apertures extend through the first member, the intermediate member, and/or second member of the pillow 10, through the first member, the intermediate member, and/or second member to thereby ventilate the pillow with cool air as illustrated by the arrows 28.

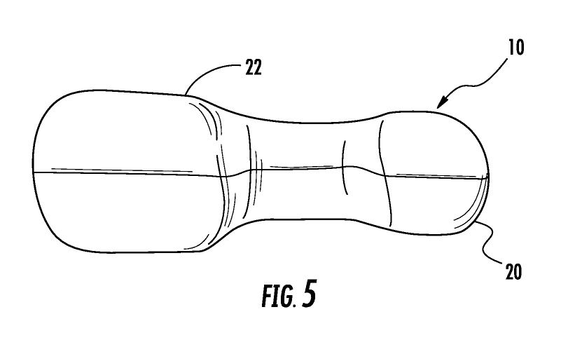

As illustrated in FIGS. 4 and 5, the first member 20 and second member 22 of the pillow 10 may be placed together such that the facing surfaces 30a and 30b are in contact with one another. The friction between the facing surfaces 30a and 30b of the first member 20 and second member 22 of the pillow 10 when the facing surfaces 30a and 30b are in contact with one another is generally sufficient, together with the cover 24, to hold the first member and second member of the pillow together during use. Similarly, in embodiments of the pillow 10 containing the intermediate member 29, such as FIG. 3A, the friction between the facing surfaces of the intermediate member 29 (not shown) and the corresponding facing surfaces 30a and 30b of the first member 20 and second member 22 of the pillow 10 is generally sufficient, together with the cover 24, to hold the first member, intermediate member and second member of the pillow together during use. Alternatively an adhesive may be used to join the corresponding facing surfaces together.

The first member 20 and second member 22 of the pillow 10 can have a variety of configurations. In one embodiment, as illustrated in FIG. 2A, the first member 20 and second member 22 of the pillow 10 are symmetrical and have substantially the same configuration. In another embodiment (not shown), the first member 20 and second member 22 of the pillow 10 are not symmetrical and/or do not have substantially the same configuration. Similarly, in embodiments of the pillow 10 containing the intermediate member 29, the first member 20, intermediate member 29, and second member 22 of the pillow 10 can have a variety of configurations. In one embodiment, as illustrated in FIG. 3A, the first member 20, intermediate member 29, and/or second member 22 of the pillow 10 are symmetrical and have substantially the same configuration. In another embodiment (not shown), the first member 20, intermediate member 29, and/or second member 22 of the pillow 10 are not symmetrical and/or do not have substantially the same configuration.

The cover 24 may be constructed of a variety of materials, including synthetic and natural fabrics and natural/synthetic blends. For purposes of example, and not limitation, the cover 24 can be constructed of silk, cotton, or polyester. The cover 24 at least partially encases the pillow 10. In one embodiment, as illustrated in FIGS. 2A, 3A, and 8, the cover 24 is at least partially constructed of a spacer fabric, also commonly referred to as 3-D mesh. Spacer fabrics have a sandwich construction and feature a first layer 32 and a second layer 34 and a third layer 36 in between the first and second layers. The inner layer 36 can take a variety of shapes and configurations, including tubes, pleats or other engineered forms. As illustrated by the arrows 38 in FIGS. 2A, 3A, and 8, the three-layer construction forms openings that enable air to flow through the first layer 32, second layer 34 and third layer 36 of the cover 24 to thereby ventilate the pillow with cool air. In one embodiment, the spacer fabric is approximately 2 to 3 mil in thickness. In one embodiment, the exterior of the cover 24 can be formed into a quilted fabric to provide a textured, three-dimensional surface. In another embodiment, the cover 24 can be constructed so as to have at least one surface containing fiberfill made of synthetic or natural fibers, or a natural/synthetic blend. In one embodiment, the at least one surface corresponds to at least one of the first member 20 and/or second member 22. In one embodiment, as illustrated in FIGS. 2A and 3A, the cover 24 has one surface containing fiberfill 25 and the surface corresponds to the first member 20 of the pillow so as to correspond to the HR urethane foam to thereby provide the user with a support surface that feels to the user more like a traditional fiberfilled pillow. The fiberfill 25 can be incorporated into the cover 24 by attaching a backing or sheet of fabric having fiberfill bonded thereto or by attaching a backing to the cover to thereby form a pocket that is then filled with the fiberfill. In either case the backing can be attached to the cover 24 by sewing or using an adhesive.

In a further embodiment, as illustrated in FIGS. 2A and 3A, the cover 24 may include a hose tether 40, which is a strap with a hook-and-loop fastener that can be used to support the weight of the air transmission hose extending from the assisted breathing or respiratory mask worn by the user to thereby reduce the pressure the weight of the hose would otherwise apply to the user's mask.

The HR urethane foam of the first member 20 provides proper ergonomic support to the user. The memory urethane foam of the second member 22 provides the user with a pliable and comfortable surface. Cradling is the ability of the urethane foam to distribute body weight uniformly over the contact area and, thus, is equivalent to surface pressure. As illustrated in the before and after illustrations in FIG. 7, it has been found that when weight is applied by the user to either the first member 20 or second member 22 of the pillow 10 of the present invention that the cradling or conforming effect is compounded because of the dual-sided design, thus providing an improved reduction in the surface pressure felt by the user. In addition, by providing a pillow 10 that includes both the comfort provided the memory urethane foam and the more ergonomic support of the HR urethane foam in a single product, the pillow of the present invention satisfies the two most popular consumer preferences of comfort and support, thereby eliminating the need of suppliers of pillows for use with assisted breathing or respiratory masks to stock pillows of differing degrees of support and comfort.

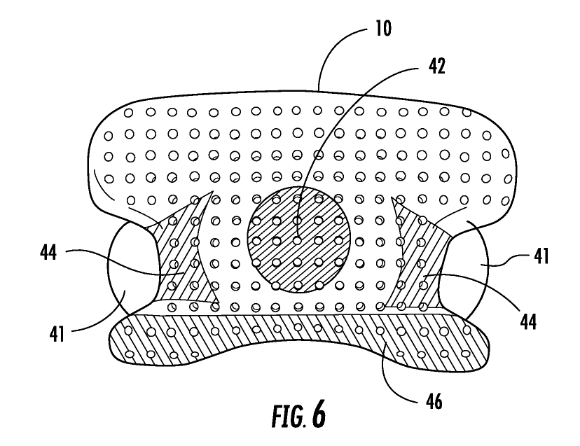

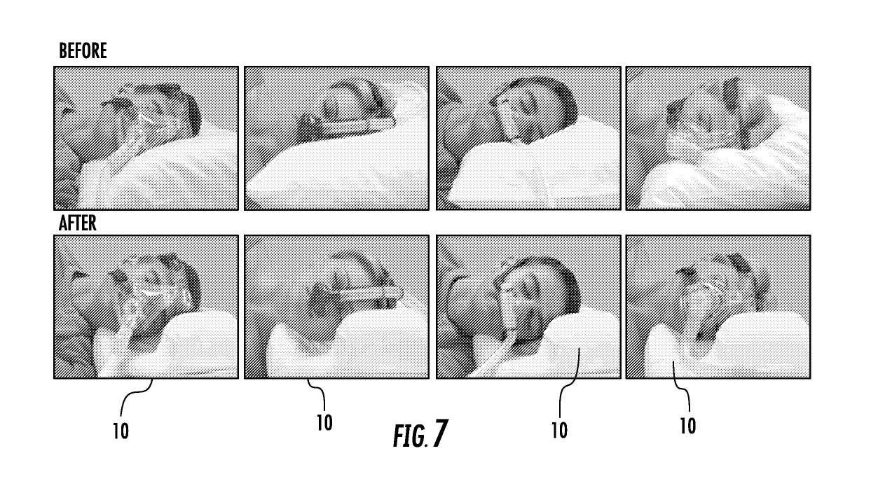

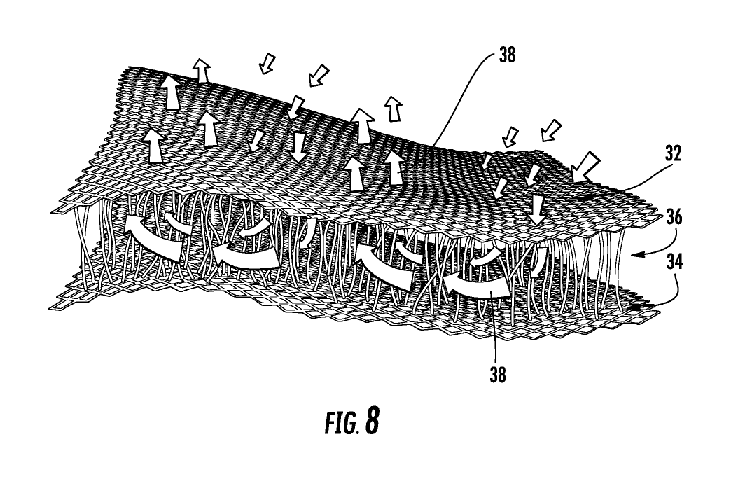

In one embodiment, as illustrated in FIG. 6, the pillow 10 may define a generally circular or oval indentation 42 in the center area of the pillow to receive and accommodate the back of a user's head thereby forming a first head landing area while the user is sleeping in the back sleeping position for optimum airway and spinal alignment. In another embodiment, also illustrated in FIG. 6, the pillow 10 may define a recess 41 on at least one of the first member 20 and second member 22 to form a pressure-free zone that prevents interference between the mask and the sleep surface when the user is sleeping in the side sleeping position so as to reduce shifting of the mask on the user's face, leaks and facial pressure to thereby improve sleep comfort and increase CPAP compliance. In a further embodiment, also illustrated in FIG. 6, the pillow 10 may define at least one second landing area 44 adjacent the recess 41 for the side sleeping position that is structured to support the cheek of the user. In a further embodiment, also illustrated in FIG. 6, the front side 14 defines a recessed leading edge to provide proper support for a user sleeping in either the side or back sleeping positions.

As noted above, the first member 20 and second member 22, and, in embodiments with an intermediate member 29, the intermediate member, include apertures 26 to enable air to flow into and, in some embodiments, through the first member 20, the intermediate member 29, and second member 22 of the pillow 10 to thereby ventilate the pillow with cool air. As further noted above, the cover 24 can be formed using a spacer fabric having a three-layer construction that includes openings that enable air to flow through the first layer 32, second layer 34 and third layer 36 of the cover 24 to thereby ventilate the pillow with cool air. The combination of the ventilation provided by the cover 24, the first member 20 and second member 22, and, in embodiments with an intermediate member 29, the intermediate member, reduces the temperature and humidity at the surface of the pillow 10 to thereby reduce perspiration from the user so that the user experiences cool and dry comfort.

Many modifications and other embodiments of the inventions set forth herein will come to mind to one skilled in the art to which these inventions pertain having the benefit of the teachings presented in the foregoing descriptions and the associated drawings.

Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A pillow for use with a respiratory mask, comprising:
first and second members, wherein the first member is made from a first material and the second member is made from a second material and wherein the first material is different from the second material.

2. A pillow according to claim 1, wherein the first material and second material are a urethane foam and wherein the first material has a lower density than the second material.

3. A pillow according to claim 1, wherein the first material and second material are a urethane foam and wherein the first material has a greater indentation load deflection than the second material.

4. A pillow according to claim 1, wherein the first member is formed of high resilient urethane foam.

5. A pillow according to claim 1, wherein the second member is formed of a memory urethane foam.

6. A pillow according to claim 1, wherein the first member defines a plurality of apertures at least partially therethrough.

7. A pillow according to claim 1, wherein the second member defines a plurality of apertures at least partially therethrough.

8. A pillow according to claim 1, wherein the first member and second member have substantially the same configuration.

9. A pillow according to claim 1, further comprising an intermediate member positioned between the first member and the second member.

10. A pillow according to claim 8, wherein the intermediate member is formed of a third material that is the same as one of the first material or the second material.

11. A pillow according to claim 8, wherein the intermediate member is formed of a third material that is different from the first material and the second material.

12. A pillow according to claim 8, wherein the intermediate member is formed of a urethane foam.

13. A pillow according to claim 8, wherein the intermediate member defines a plurality of apertures at least partially therethrough.

14. A pillow according to claim 8, wherein the intermediate member and at least one of the first member and second member have substantially the same configuration.

15. A pillow according to claim 1, further comprising a cover that at least partially covers the first and second members.

16. A pillow according to claim 14, wherein at least a portion of the cover is formed of a spaced fabric.

17. A pillow according to claim 13, wherein the cover comprises a fiberfilled surface on at least one side.

18. A pillow according to claim 15, wherein the fiberfilled surface is positioned adjacent the first member.

19. A pillow according to claim 13, wherein the cover comprises a hose tether extending therefrom.

20. A method of using a respiratory mask, comprising:
providing a pillow having first and second members, wherein the first member is made of a first material and the second member is made from a second material and wherein the first material is different from the second material;
positioning a respiratory mask on the head of a user; and
supporting the user's head on the pillow.
